(19) United States
(12) Patent Application Publication
(10) Pub. No.: US 2013/0072988 A1
(43) Pub. Date: Mar. 21, 2013

(54) Aiming on Plate

(71) Applicant: Urs Hulliger, Oberdorf (CH)

(72) Inventor: Urs Hulliger, Oberdorf (CH)

(21) Appl. No.: 13/426,079

(22) Filed: Mar. 21, 2012

Related U.S. Application Data
(60) Provisional application No. 61/487,466, filed on May 18, 2011.

(51) Int. Cl. A61B 17/80

(52) U.S. Cl. CPC A61B 17/808
USPC 606/281; 606/280

(57) ABSTRACT

A bone fixation device includes an aiming guide extending from a first end to a second end, the aiming guide having a plurality of aiming holes, each aiming hole extending through the aiming guide from a proximal face to a distal face along a corresponding aiming hole axis and a bone implant having a proximal face facing the aiming guide and a distal face which faces a target portion of bone, the implant including a plurality of implant holes each extending through the implant from the proximal face to the distal face, an implant hole axis of each of the implant holes being aligned with the aiming hole axis of a corresponding one of the aiming holes. A first connecting bar is integrally formed with the aiming guide and bone implant to space the distal face of the aiming guide from the proximal face of the implant.

### PRIORITY CLAIM

The present application claims priority to U.S. Provisional Application Ser. No. 61/487,466 filed on May 18, 2011 and entitled “Aiming on Plate” to Urs Hulliger, the entire disclosure of which is incorporated herein by reference.

### FIELD OF THE INVENTION

The present invention is related to the field of bone fixation and, more specifically, to a system and method for the alignment of a bone fixation device (e.g., a bone plate) over a fractured or otherwise damaged bone.

### BACKGROUND INFORMATION

Procedures for the fixation of fractured or otherwise damaged bone often involve the placement of a bone plate over a target portion of the bone. Once the bone plate has been positioned over the bone, an aiming device is inserted into the body and secured to one of the bone and the bone plate in a target position. A drill guide is then inserted through the aiming device and into the bone plate to guide a drilling device therethrough to drill a hole in the bone. One or more bone fixation elements (e.g., bone screws, intramedullary rods, etc.) are then guided through holes in the bone plate hole into the bone to fix the plate to the bone. Current devices and methods generally require the bone plate to be inserted separately from the aiming device, complicating and lengthening these procedures.

### SUMMARY OF THE INVENTION

The present invention relates to a bone fixation system comprising an aiming guide extending from a first end to a second end, the aiming guide having a plurality of aiming holes, each aiming hole extending through the aiming guide from a proximal face to a distal face thereof along a corresponding aiming hole axis. The system further comprises a bone implant extending from a third end to a fourth end and having a proximal face facing the aiming guide and a distal face which, when in a desired position, faces a target portion of bone to which the implant is to be coupled, the implant including a plurality of implant holes each extending through the implant from the proximal face to the distal face thereof along a corresponding implant hole axis, the implant hole axis of each of the implant holes being aligned with the aiming hole axis of a corresponding one of the aiming holes and a first connecting bar integrally formed with the aiming guide and bone implant to space the distal face of the aiming guide from the proximal face of the implant.

### BRIEF DESCRIPTION OF THE DRAWINGS

FIG. 1 shows a first perspective view of a bone fixation system according to an exemplary embodiment of the present invention in a first operative configuration;

FIG. 2 shows a second perspective view of the device of FIG. 1 in a second operative configuration;

FIG. 3 shows a third perspective view of the device of FIG. 1 in a third operative configuration;

FIG. 4 shows a fourth perspective view of the device of FIG. 1 in a fourth operative configuration; and

FIG. 5 shows a cross-sectional view of the device of FIG. 1.

### DETAILED DESCRIPTION

The present invention may be further understood with reference to the following description and the appended drawings. The present invention relates generally to devices and methods for the fixation and stabilization of long bone fractures. It is noted that although embodiments of the present invention are described with respect to particular bones, the present invention may be employed in a variety of other bone fixation procedures as would be understood by those skilled in the art. The present invention relates to an aiming device configured to guide the insertion of a bone fixation element (e.g., a bone screw) into a fractured or otherwise damaged bone. The exemplary aiming guide according to the present invention is preferably formed to correspond to a bone plate hole axis of a bone plate hole or screw holes formed in the bone plate through which bone fixation elements (e.g., bone screw and/or pins) are to be inserted. One or more guide holes extend through the aiming guide. The guide holes are in alignment with the bone plate hole axes extending through the bone plate in order to guide the bone fixation element through the bone plate holes into the bone at desired angles, as will be described in greater detail hereinafter. The exemplary aiming guide according to the invention is connected to the bone plate via one or more connecting bars, which may, for example, be integrally manufactured with the bone plate and aiming guide, as will be described in greater detail hereinafter. The term proximal, as used herein, refers to a direction approaching a physician or other user while the term distal refers to a direction approaching a target portion of a fractured or otherwise damaged bone.

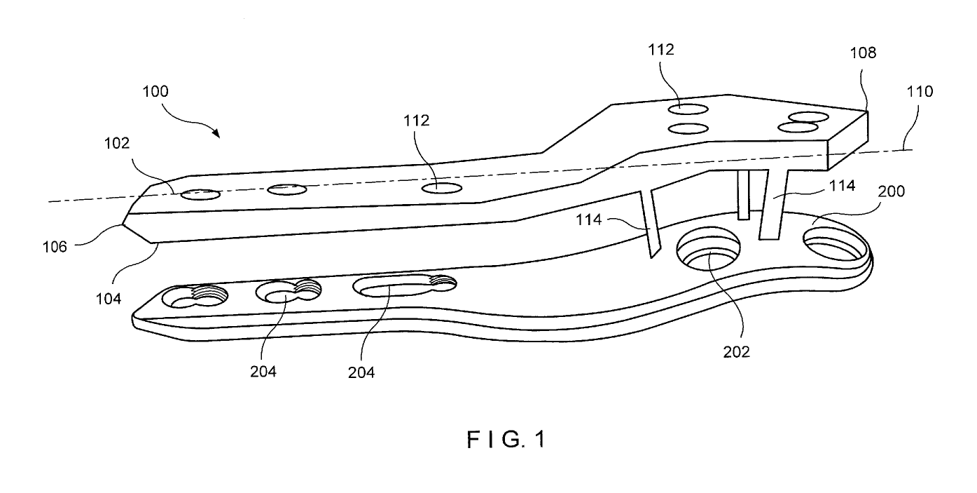

As described in FIGS. 1-5, the present invention is directed to an aiming guide 100 integrally formed with a bone implant 200 (e.g., a bone plate) configured for insertion into a living body. The aiming guide 100 has a proximal face 102 and a distal face 104 and extends from a first end 106 to a second end 108 along a longitudinal axis 110. The aiming guide 100 may be any shape, size and curvature provided that guide holes 112 extending through the aiming guide 100 are in alignment with corresponding bone implant plate holes 202 in the bone implant 200. In the exemplary embodiment shown in FIG. 1, the aiming guide 100 has a shape and curvature that conforms substantially to the shape and curvature of the bone implant 200. In other exemplary embodiments, the shape and curvature of the aiming guide 100 could be defined according to a user's preferences as long as the aiming guide 100 accommodates the required positioning, dimensions and angulations of the guide holes 112. For example, the shape of the aiming guide 100 corresponds to a major axis of the bone implant 200 for indicating the direction of insertion of the bone implant 200.

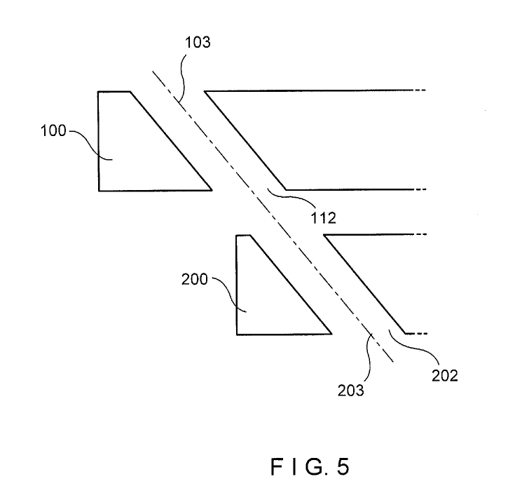

The aiming guide 100 comprises the plurality of aiming guide holes 112 extending therethrough from the proximal face 102 to the distal face 104. The guide holes 112 align with the holes 202 in the bone implant 200 so that bone fixation elements may be inserted into a guide holes 112 and guided into a corresponding one of the bone implant holes 202 in the bone implant 200. As shown in FIG. 5, a central axis 103 of the guide holes 112 may be coaxial with a central axis 203 of the bone implant hole 202. In a first exemplary embodiment, the aiming guide holes 112 extend substantially perpendicularly to the proximal and distal faces 102, 104 to align with substantially perpendicular bone implant holes 202 extending through the bone implant 200. Where the bone implant 200 is provided with a combination hole 204, the aiming guide hole 112 may be positioned so that a drill guide inserted therethrough aligns with a desired portion of the combination hole. In another embodiment, the aiming guide hole 112 may be angled to guide a bone fixation element (e.g., a bone screw, etc.) inserted through the bone implant 200 and into a bone at a predefined non-perpendicular angle relative to the proximal and distal faces 102, 104. In yet another embodiment (not shown), the aiming guide hole 112 may also be formed as a combination hole permitting the insertion of a bone fixation element into the bone at a user-selected angle to conform to the requirements of a particular bone fixation procedure. As those skilled in the art will understand, where an angled insertion is required, the aiming guide hole 112 may be longitudinally offset from the bone implant hole 202 by a distance selected to permit alignment of the bone fixation elements with each of the holes 112, 202.

The aiming guide 100 may, for example, be manufactured of the same material as the bone implant 200 as a single element, and may be separated from the bone implant 200 by a plurality of connecting bars 114. The aiming guide 100 according to the exemplary embodiment of FIGS. 1-5 includes three connecting bars 114. It is noted, however, that any number of connecting bars 114 may be used provided that the aiming guide 100 remains connected to the bone implant 200 until a disconnection operation is performed, as will be described in greater detail below. Placement of the connecting bars 114 on the aiming guide 100 may be selected such that, once inserted to a desired position within the body, the connecting bars 114 are positioned adjacent a minimally invasive incision formed in the body, as will be described in greater detail later on. The length of connecting bars 114 may range from 1 cm up to 10 cm. The length is chosen according to the location in the body in which the bone implant 200 is to be inserted. The following are exemplary ranges of length of the connecting bars 114 according to where in the body a bone implant 200 is to be used:
Shoulder: 3 to 5 cm.;
Elbow: 2 to 3 cm.;
Hand and wrist: 1 to 2 cm.;
Hips and pelvis: 5 to 10 cm.;
Knee: 5 to 8 cm.;
Ankle: 3 to 5 cm.; and
Foot: 2 to 3 cm.

The connecting bars 114 may also be formed of the same material as the bone implant 200 and aiming guide 100 or of any other biocompatible suitably rigid material as would be understood by those skilled in the art. For example, as will be described in more detail below, the aiming guide 100 and the bone implant 200 may be milled from a single piece of material and separated from one another after the aiming guide 100 has been used to insert bone fixation elements through the bone implant 200 into the bone 10.

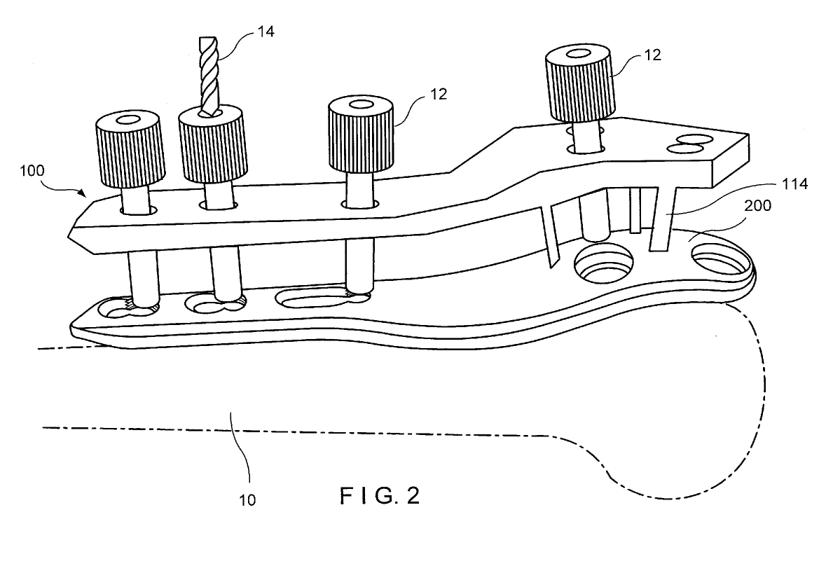

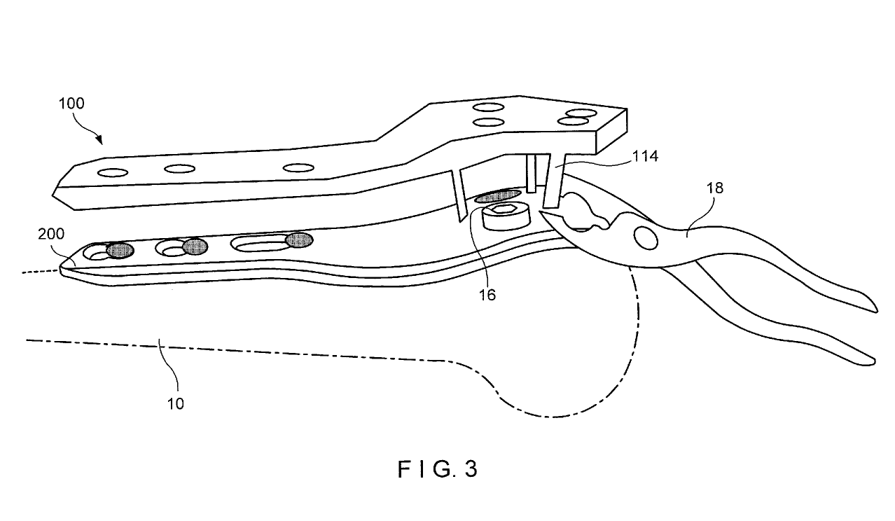

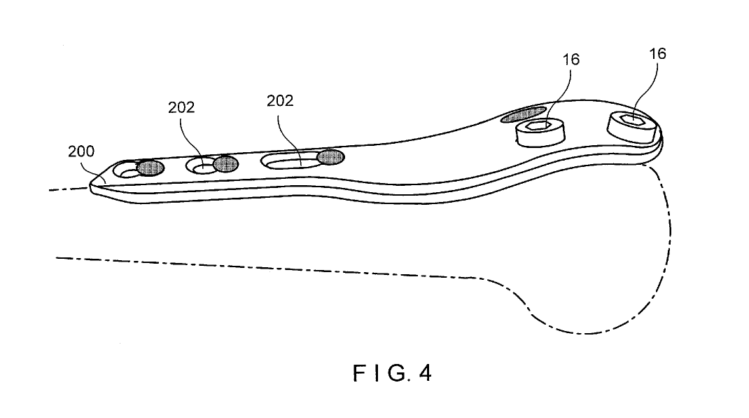

As shown in FIGS. 2-4, an exemplary method according to the invention includes insertion of the bone implant 200 and the aiming guide 100 through a minimally invasive incision to a desired position over a target portion of a bone 10 to which the bone implant 200 is to be coupled. One or more drill guides 12 are then inserted through the aiming guide holes 212 into the bone implant holes 202, each at a desired angle at which a bone fixation element is to extend through the corresponding bone implant hole 202 into the bone 10. As shown in FIG. 3, after all of the drill guides 12 have been properly positioned, for each of the drill guides 12, a physician or other user inserts a drill 14 through a bore extending through the drill guide 12 and through the corresponding bone implant hole 202 to drill a bore in the bone 10. After all of the holes have been drilled into the bone 10, a bone fixation element (e.g., a bone screw, bone pin, etc.) 16 is inserted into each of the drilled bores to secure the bone implant 200 to the bone 10. Once the bone implant 200 has been secured, the physician or other user performs a disconnection operation to separate the aiming guide 100 from the bone implant 200. The disconnection operation may use a clipping device 18 known in the art to clip each of the connecting bars 114 to separate the aiming guide 100 from the bone implant 200. As those skilled in the art will understand, the connecting bars 114 are clipped so that any remaining portion thereof lies flush against a proximal face 206 of the bone implant 200. As shown in FIG. 4, the aiming device 100 and the clipped portions of the connecting bars 114 are then removed from the body. It will be understood by those of skill in the art that the disconnection operation may not remove all of the connecting bars 114 and a further operation, such as filing, may be required to smooth the surface of the proximal face 206.

The aiming guide 100 and bone implant 200 may be made using a known manufacturing technique. Such a technique includes, but is not limited to milling, laser sintering, molding, casting or welding together two separately formed components. In an exemplary manufacturing method, the aiming guide 100 and bone implant 200 are manufactured and subsequently packaged as a single piece. For example, where milling is used, the single piece is manufactured from a single block of material. In another example, where laser sintering is used, the single piece is formed by building up layers of the material in a desired shape and subsequently milling the bone implant holes 202 when necessary. The shape and configuration of the aiming guide 100 and bone implant 200 may be patient-specific or may correspond to a standard implant.

It will be apparent to those skilled in the art that various modifications and variations may be made in the structure and the methodology of the present invention, without departing from the spirit or scope of the invention. Thus, it is intended that the present invention cover modifications and variations of the invention provided that they come within the scope of the appended claim's and their equivalents.

What is claimed is:

1. A bone fixation device, comprising a bone implant integrally formed with an aiming guide, the aiming guide being spaced apart from the bone implant by a first connecting bar extending therebetween.

2. The bone fixation device of claim 1, further comprising a second connecting bar connecting the aiming guide to the implant.

3. The bone fixation device of claim 1, wherein the bone implant includes a plurality of implant holes, each of the implant holes being configured to receive a bone fixation element to couple the bone implant to a target portion of the bone and wherein the aiming guide comprises a plurality of aiming holes, each of the aiming holes being aligned with a corresponding one of the implant holes so that a drill guide inserted through a first one of the aiming holes along a first aiming hole axis of the first aiming hole is aligned with a first implant hole axis of a corresponding first one of the implant holes along which a first bone fixation element is to be inserted through the implant into the target portion of bone.

4. The bone fixation device of claim 3, wherein a second one of the aiming holes extends through the aiming guide from the proximal face to the distal face along a second aiming hole axis aligned with a second implant hole axis along which a bone fixation element is to be inserted through a second one of the implant holes into the target portion of bone.

5. The bone fixation device of claim 1, wherein the first connecting bar is positioned so that, when the aiming guide and implant are in a desired position over the target portion of bone, the first connecting bar is accessible to separate the aiming guide from the bone implant.

6. A bone fixation system, comprising:
an aiming guide extending from a first end to a second end, the aiming guide having a plurality of aiming holes, each aiming hole extending through the aiming guide from a proximal face to a distal face thereof along a corresponding aiming hole axis;
a bone implant extending from a third end to a fourth end and having a proximal face facing the aiming guide and a distal face which, when in a desired position, faces a target portion of bone to which the implant is to be coupled, the implant including a plurality of implant holes each extending through the implant from the proximal face to the distal face thereof along a corresponding implant hole axis, the implant hole axis of each of the implant holes being aligned with the aiming hole axis of a corresponding one of the aiming holes; and
a first connecting bar integrally formed with the aiming guide and bone implant to space the distal face of the aiming guide from the proximal face of the implant.

7. The bone fixation system of claim 7, further comprising a second connecting bar spacing the distal face of the aiming guide from the proximal face of the implant.

8. A method for bone fixation, comprising the steps of:
inserting to a desired position over target portion of bone in a living body a bone fixation device comprising:
an aiming guide extending from a first end to a second end, the aiming guide having a plurality of aiming holes each extending through the aiming guide from a proximal face to a distal face of the aiming guide to a distal face thereof along a corresponding aiming hole axis;
a bone implant extending from a third end to a fourth end and having a proximal face facing the distal face of the aiming guide and a distal face which, when in a desired position, faces a target portion of bone to which the implant is to be coupled, the implant including a plurality of implant holes each extending through the implant from the proximal face to the distal face thereof along a an implant hole axis aligned with the aiming hole axis of a corresponding one of the aiming holes; and
a first connecting bar spacing the distal face of the aiming guide from the proximal face of the implant;
fixing the bone implant to the bone by inserting a bone fixation element into a target portion of bone via a first one of the implant holes; and
disconnecting the first connecting bar from the bone implant at a location substantially flush with the proximal face of the bone implant to separate the aiming guide from the implant.

9. The method according to claim 8, further comprising the step of removing the aiming guide and first connecting bar from the body.

10. The method according to claim 8, wherein the step of fixing further comprises:
inserting a drill guide through the first aiming guide hole into the first implant hole;
inserting a drill through the drill guide to drill a bore into the bone along the first implant hole axis; and
inserting a bone fixation element into the bore to secure the bone implant to the bone.

11. The method according to claim 8, wherein the step of disconnecting further comprises clipping with a clipping device the first connecting bar at a location substantially flush with the proximal face of the bone implant to separate the aiming guide from the bone implant.

12. A method for manufacturing a bone fixation device, comprising the steps of integrally forming a bone fixation device including a bone implant, an aiming guide and a connecting bar connecting the bone implant to the aiming guide.

13. The method of claim 12, wherein the bone fixation device is formed by one or more of milling, laser sintering, molding, casting or welding together two separately formed components.

14. The method of claim 12, wherein the bone fixation device is milled from a single block of material.

15. The method of claim 14, wherein the block comprises a plurality of individual layers of material.

16. The method of claim 12, further comprising the step of milling bone implant holes in the bone implant.

17. The method of claim 16, further comprising the step of milling a plurality of aiming holes in the aiming guide, an aiming hole axis of each of the aiming holes being aligned with an implant hole axis of a corresponding one of the implant holes to guide bone fixation elements inserted through the aiming holes into the implant holes to extend therethrough into a target portion of bone.
